(19) United States
(12) Patent Application Publication
(10) Pub. No.: US 2020/0390664 A1
(43) Pub. Date: Dec. 17, 2020

(54) COSMETIC FILM

(71) Applicant: The Honest Company, Inc., Los Angeles, CA (US)

(72) Inventors: Mallory McMahon, Marina Del Rey, CA (US); Kevin Ewell, Simi Valley, CA (US); Sarah Anjuwon, Gardena, CA (US)

(21) Appl. No.: 16/899,926

(22) Filed: Jun. 12, 2020

Related U.S. Application Data
(60) Provisional application No. 62/861,603, filed on Jun. 14, 2019.

(51) Int. Cl. A61K 8/02; A61K 8/31; A61K 8/36; A61K 8/362; A61K 8/365; A61K 8/92; A61K 8/73; A61Q 1/06

(52) U.S. Cl. CPC A61K 8/0229; A61K 8/31; A61K 8/361; A61K 8/362; A61K 2800/43; A61K 8/922; A61K 8/735; A61Q 1/06; A61K 8/365

(57) ABSTRACT

A cosmetic formulation including a plant derived volatile emollient, a plant derived film former, a pliable wax, and a hyaluronic acid, that provides long lasting wear and increased skin hydration for at least 6 hours.

### BACKGROUND

The present invention generally relates to cosmetic products, and more particularly to lipstick, including liquid and solid lipsticks.

### SUMMARY

In accordance with an embodiment of the present invention, a cosmetic formulation is provided. The cosmetic formulation includes a plant derived volatile emollient, a plant derived film former, and a pliable wax.

In accordance with another embodiment of the present invention, a cosmetic formulation is provided. The cosmetic formulation includes a plant derived volatile emollient, a plant derived film former, a pliable wax, and a hyaluronic acid.

In accordance with yet another embodiment of the present invention, a cosmetic formulation is provided. The cosmetic formulation includes a plant derived volatile emollient, wherein the plant derived volatile emollient is an emollient derived from a plant, fruit, or vegetable source having a vapor pressure greater than 0.13 Pascal (Pa). In other embodiments, the plant derived volatile emollient has a flash point below 100° C., and can also have a boiling point below 230° C. The cosmetic formulation further includes a plant derived film former. The cosmetic formulation further includes a pliable wax, wherein the pliable wax is an organic compound or mixture of organic compounds that is a non-crystalline solid at about 25° C. The pliable wax may also have a melting point in a range of about 45° C. to about 100° C., and/or a penetration value, as measured using ASTM D1321, greater than 40 dmm (decimillimeters) at 25° C. The cosmetic formulation further includes an oil dispersible hyaluronic acid, and a colorant.

In accordance with yet another embodiment of the present invention, a cosmetic formulation is provided. The cosmetic formulation includes a plant derived volatile emollient, wherein the plant derived volatile emollient is one selected from a group consisting of coconut alkanes, undecane (C₁₁H₂₄), dodecane (C₁₂H₂₆), tridecane (C₁₃H₂₈), and combinations thereof. The cosmetic formulation further includes a plant derived film former, wherein the plant derived film former is one selected from a group consisting of glyceryl rosinate, hydrogenated glyceryl abietate, capryloyl glycerin/sebacic acid copolymer, and combinations thereof. The cosmetic formulation further includes a pliable wax, wherein the pliable wax is one selected from a group consisting of a polyhydroxystearic acid, C₁₈-C₃₈ alkyl hydroxystearoyl stearate, and combinations thereof. The cosmetic formulation further includes an oil dispersible hyaluronic acid.

These and other features and advantages will become apparent from the following detailed description of illustrative embodiments thereof, which is to be read in connection with the accompanying drawings.

### BRIEF DESCRIPTION OF THE DRAWINGS

The following description will provide details of preferred embodiments with reference to the following figures wherein:

FIGS. 1 and 2 are a block/flow diagram of a method of preparing a cosmetic formulation for a liquid lipstick, in accordance with an embodiment of the present invention;

FIGS. 3 and 4 are another block/flow diagram of a method of preparing a cosmetic formulation for a solid lipstick, in accordance with an embodiment of the present invention; and

FIGS. 5 and 6 are yet another block/flow diagram of a method of preparing a cosmetic formulation for a liquid foundation, in accordance with an embodiment of the present invention.

### DETAILED DESCRIPTION

Aspects of the present invention relate generally to a novel way to create long lasting, comfortable cosmetic products with greener, sustainable film forming components using a plant derived volatile emollient, a plant derived film former, a pliable wax. The use of a plant derived volatile emollient and plant derived film former in combination with a pliable wax can create a comfortable, long lasting cosmetic formulation. This also contributes to achieving long wear in a cosmetic formula without the use of petroleum derived emollients or synthetic film formers. Many cosmetic products can claim to have long wear or lasting color. These products, however, can be uncomfortable to wear long term and can be drying to the skin due to volatile silicones or petroleum derived emollients or synthetic film formers.

Aspects of the present invention relate generally to a novel way to create long lasting, comfortable cosmetic products with greener, sustainable film forming components using a plant derived volatile emollient, a plant derived film former, a pliable wax, and an oil dispersible hyaluronic acid. The use of a plant derived volatile emollient and plant derived film former in combination with a pliable wax and oil dispersible hyaluronic acid can create a comfortable, moisturizing, long lasting cosmetic formulation. This also contributes to achieving long wear in a cosmetic formula without the use of petroleum derived emollients or synthetic film formers.

Embodiments of the present invention can provide moisturizing cosmetics that are more comfortable to wear and have longer lasting color than products containing volatile silicones, petroleum derived emollients, and/or synthetic film formers. Replacement of silicone and petroleum based components with greener, sustainable components that come from plant based renewable sources can improve the feel and provide increased moisturizing characteristics.

Embodiments of the present invention provide moisturizing cosmetics including a plant derived volatile emollient and a plant derived film former that has long wear, water resistance, and substantivity to the skin. Utilizing plant based film formers makes formulations that have long wear and sustainability more difficult to achieve than silicone and/or petroleum based products. Use of a pliable wax which functions as a film plasticizer and enhances film flexibility and formula stability in combination with the plant based film former(s) can overcome this difficulty.

For the purpose of this invention, long wear is defined as 6 hours or more of product wear before a consumer requires reapplying for aesthetic purposes, where a statistically significant portion of a test panel exhibited 80% or better coverage after 6 hours of wear.

For the purpose of this invention, water resistance is defined as having less than 50% decrease in either color saturation or (for Sunscreen products) SPF after 80 minute immersion testing per FDA 2011 21 CFR 201310/ISO 24444:2010.

For the purpose of this invention, cosmetic colorants include any pigments subject to the Food, Drug, and Cosmetic Act of 1938 (FD&C Act), sec. 721; 21 U.S.C. 379e, dyes, pigments, or other substances that can impart color when added or applied to a food, drug, cosmetic, or the human body.

Long lasting refers to at least 6 hours (hrs), at least 8 hrs, at least 12 hrs, at least 18 hrs, or at least 24 hrs.

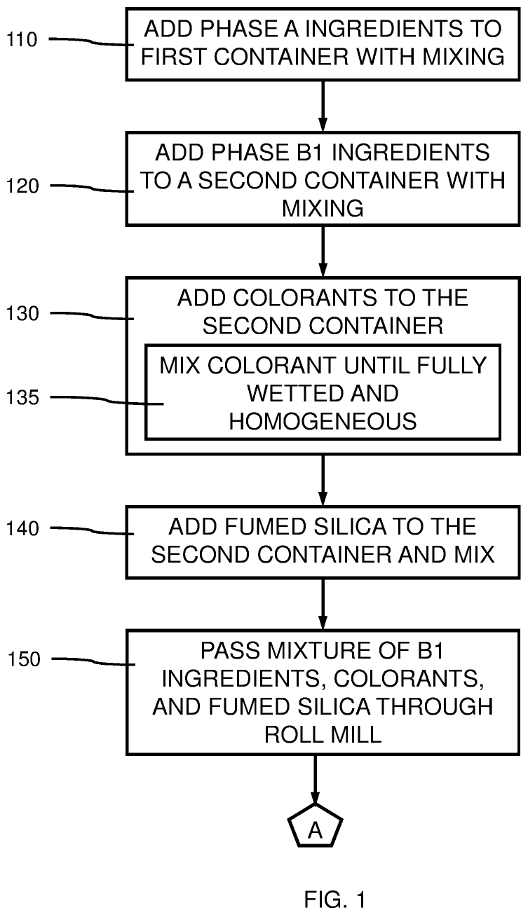

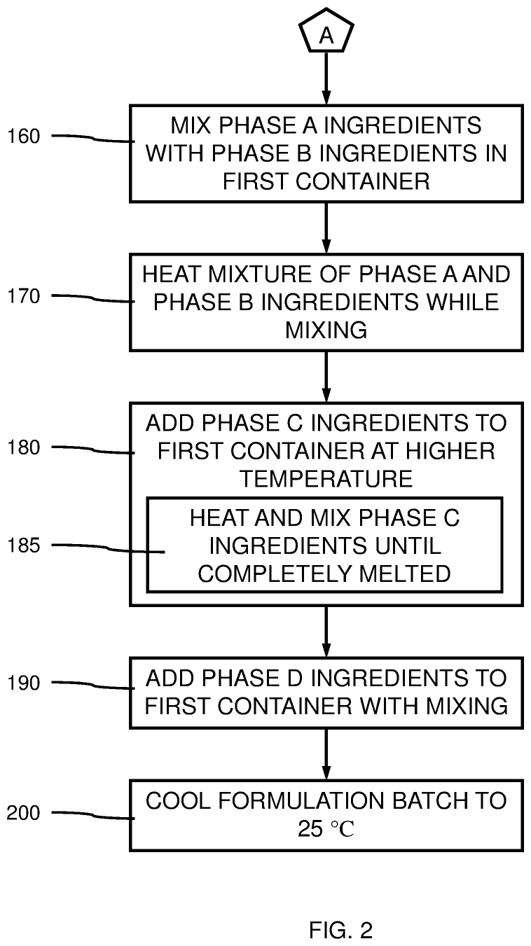

Referring now to the drawings in which like numerals represent the same or similar elements and initially to FIGS. 1 and 2, a method of preparing a cosmetic formulation for a liquid lipstick is shown, in accordance with an embodiment of the present invention.

In block 110, Phase A ingredients are placed in a main kettle that can be closed. The Phase A ingredients can be mixed during addition of the individual ingredients, as well as after all ingredients have been added to the kettle.

In block 120, the B1 ingredients can be added to a side kettle, where the Phase B1 ingredients can be mixed until homogeneous.

In block 130, the B2 ingredients, which can be colorants, can be added to the homogeneous mixture of B1 ingredients in the side kettle.

In block 135, the B1 and B2 ingredients can be mixed until the colorants are fully wetted and dispersed throughout the mixture.

In block 140, the B3 ingredients can be added to the mixture of B1 and B2 ingredients, where the B3 ingredients can be silica acting as a filler.

In block 150, the mixture of B1, B2, and B3 can be passed through a roll mill to disperse the colorants. The roll mill can be a three roll mill. The mixture of B1, B2, and B3 can be passed through the roll mill multiple times. In various embodiments, the mixture of B1, B2, and B3 can be passed through the three roll mill at least three times.

In block 160, the Phase B mixture of B1, B2, and B3 can be added to the Phase A mixture in the main kettle.

In block 170, the mixture of Phase A and Phase B ingredients in the main kettle can be heated to a predetermined temperature. In various embodiments, the temperature can be in a range of about 60° C. to about 100° C., or about 90° C. The Phase A and Phase B ingredients in the main kettle can be mixed while heating, where the mixing can be continuous.

In block 180, the Phase C ingredients can be added to the main kettle, when the mixture in the main kettle is at the predetermined temperature, for example, about 90° C.

In block 185, the mixture of Phase A, Phase B, and Phase C ingredients are mixed at the elevated temperature until the Phase C ingredients are melted.

In block 190, the Phase D ingredients can be added to the main kettle, where the Phase D ingredients can be mixed with the ingredients in the main kettle for a predetermined period of time, for example, 10 minutes.

In block 200, the melted mixture of ingredients in the main kettle can be removed from the main kettle and allowed to cool. The ingredients can be allowed to cool to 25° C.

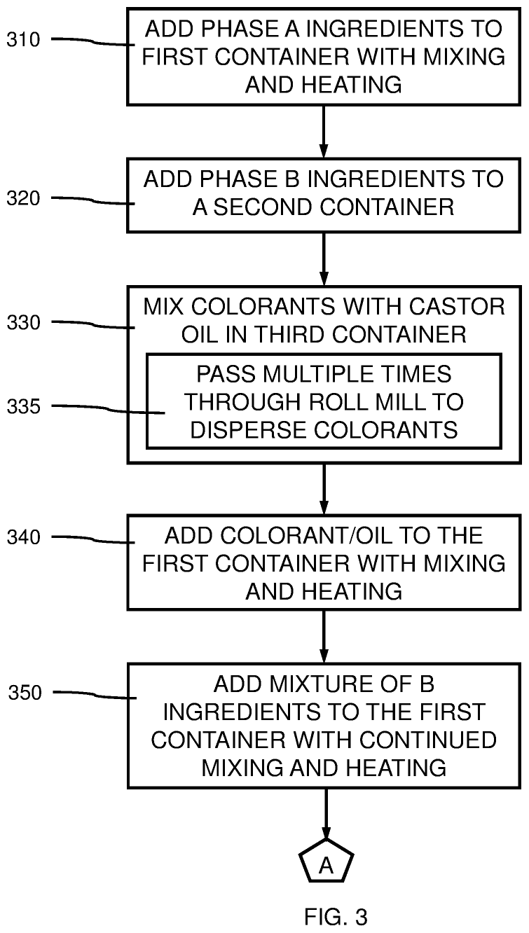

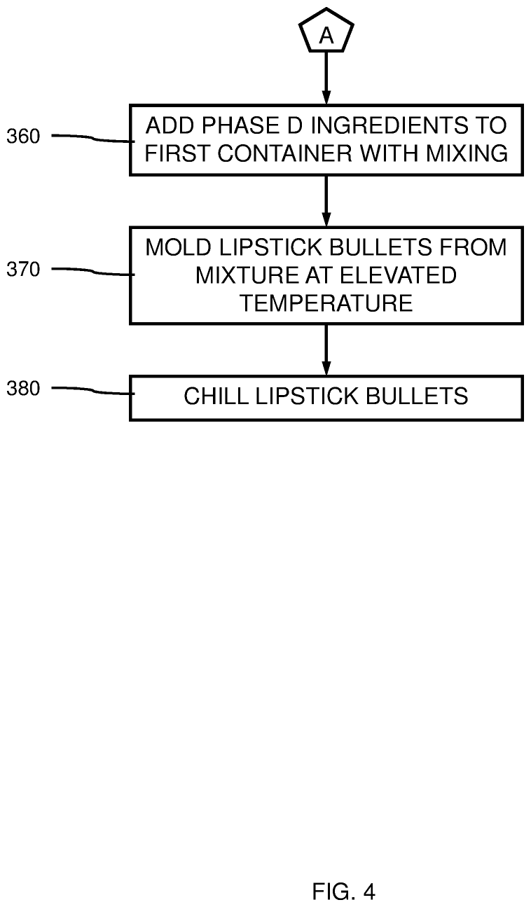

FIGS. 3 and 4 are another block/flow diagram of a method of preparing a cosmetic formulation for a solid lipstick, in accordance with an embodiment of the present invention. Solid lipsticks and liquid lipsticks may be referred to collectively as lipsticks.

In block 310, Phase A ingredients are placed in a main kettle that can be closed. The Phase A ingredients can be mixed during addition of the individual ingredients, as well as after all ingredients have been added to the kettle. The Phase A ingredients in the main kettle can be heated while mixing to a predetermined temperature. In various embodiments, the temperature can be in a range of about 60° C. to about 100° C., or about 90° C.

In block 320, the Phase B ingredients can be added to a side kettle. This can be done while the main kettle is heating to an elevated temperature.

In block 330, the Phase C ingredients, including the colorants, can be mixed with castor oil and passed through a roll mill to disperse the colorants. The roll mill can be a three roll mill. The mixture of colorants and castor oil can be passed through the roll mill multiple times. In various embodiments, the mixture of Phase C ingredients can be passed through the three roll mill at least three times.

In block 340, the mixture of Phase C ingredients can be added to the main kettle, where mixing and heating can be continued. The mixture of Phase A and Phase C ingredients can be brought to an elevated temperature. In various embodiments, the temperature can be about 90° C.

In block 350, the Phase B mixture can be added to the main kettle, while the main kettle is at the elevated temperature. The mixture of Phase A, B, and C can be mixed at the elevated temperature until all of the waxes are melted and dispersed.

In block 360, the Phase D ingredients can be added to the main kettle and mixed in. The mixing can continue until the ingredient mixture is homogeneous.

In block 370, lipstick bullets can be molded from the mixture of Phase A, B, C, and D, while at the elevated temperature.

In block 380, the temperature of the molded lipstick bullets can be reduced. In various embodiments, the molded lipstick bullets can be chilled to 50° C., or to room temperature (i.e., 25° C.).

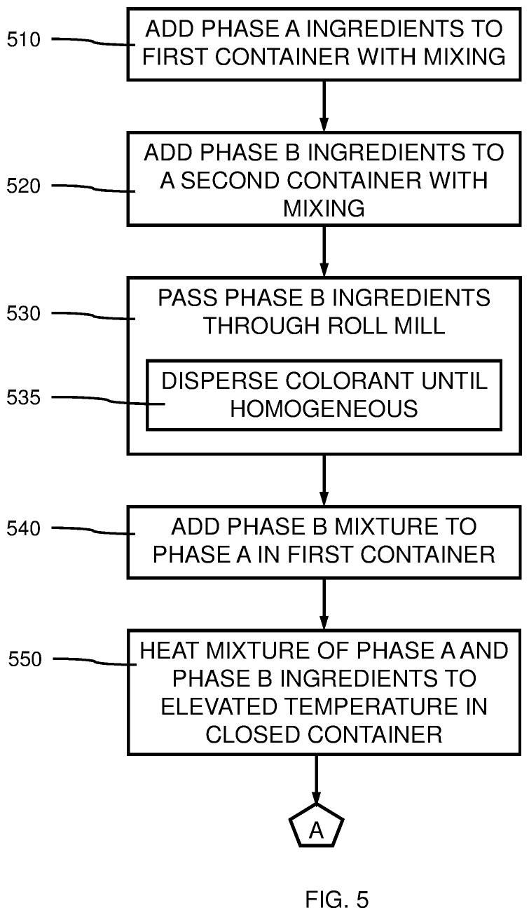

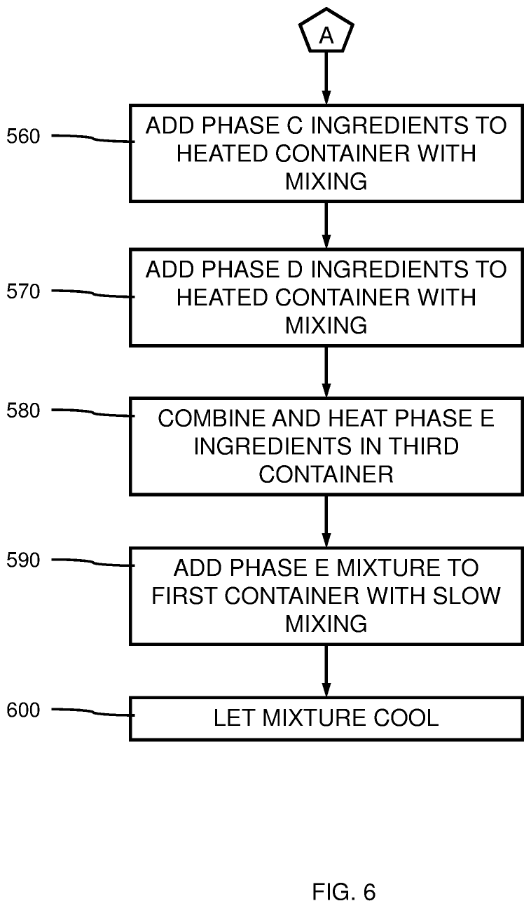

FIGS. 5 and 6 are yet another block/flow diagram of a method of preparing a cosmetic formulation for a liquid foundation, in accordance with an embodiment of the present invention.

In block 510, Phase A ingredients are placed in a main kettle that can be closed. The Phase A ingredients can be mixed during addition of the individual ingredients, as well as after all ingredients have been added to the kettle.

In block 520, the Phase B ingredients can be added to a side kettle with mixing The mixing can be continuous.

In block 530, the mixed Phase B ingredients can be passed through a roll mill. The roll mill can be a three roll mill. The mixture of Phase B ingredients can be passed through the roll mill multiple times. In various embodiments, the mixture of Phase B ingredients can be passed through the three roll mill at least three times.

In block 540, the mixture of Phase B ingredients can be added to the Phase A ingredients in the main kettle.

In block 550, the mixture of Phase A and Phase B ingredients in the main kettle can be mixed and heated. The Phase A and Phase B ingredients in the main kettle can be heated to a temperature in a range of about 40 C to about 90 C, or about 85 C. The main kettle can be closed during heating.

In block 560, the Phase C ingredients can be added to the Phase A and Phase B ingredients at the elevated temperature.

In block 570, the Phase D ingredients can be added to the Phase A and Phase B ingredients at the elevated temperature.

In block 580, the Phase E ingredients can be added to a third kettle, and heated to an elevated temperature. The Phase E ingredients can be heated to a temperature in a range of about 40 C to about 90 C, or about 85 C.

In block 590, the Phase E ingredients can be added to the mixture of A, B, C, and D ingredients in the main kettle at the elevated temperature. The Phase E ingredients can be added to the mixture of A, B, C, and D ingredients with slow mixing.

In block 600, once all of Phase E was added, heat can be removed and the batch mixed until the temperature reached 30° C. The formula can be homogenized for 5 minutes to complete the batch.

In one or more embodiments, the cosmetic formulation can include a plant derived volatile emollient, a plant derived film former, a pliable wax, and an oil dispersible hyaluronic acid.

In various embodiments, the plant derived volatile emollient can be an emollient derived from a plant, fruit, or vegetable source with a vapor pressure greater than 0.13 Pa, a flash point below 100° C., and a boiling point below 230° C. The volatile emollient can assist in setting the film and wax components quickly on a surface (e.g., lips), where the volatile emollient is a liquid at room temperature (i.e., 25° C.) and quickly vaporizes leaving the film former and pliable wax components behind. The volatile emollient may not absorb into the skin but evaporates from it.

In one or more embodiments, the plant derived volatile emollient can be coconut alkanes, undecane (C₁₁H₂₄), dodecane (C₁₂H₂₆), tridecane (C₁₃H₂₈), and combinations thereof. The coconut alkanes can be a mixture with coco-caprylate caprate, where the mixture can be present in the formulation in the described weight percent range. The coconut alkanes can make up at least 80 wt. % to 100 wt. %, or at least 90 wt. % to 100 wt. % of the mixture with the coco-caprylate caprate.

In various embodiments, the plant derived volatile emollient can be present in the formulation in a range of about 1.0 weight-percent (wt. %) to about 80.0 wt. %, or about 10.0 wt. % to about 70.0 wt. %, or about 15.0 wt. % to about 50.0 wt. %, although other ranges are also contemplated. In various embodiments, the plant derived volatile emollient(s) can make up the balance of the cosmetic formulation after all other components are included.

In one or more embodiments, the pliable wax can be an organic compound or mixture of organic compounds that is a non-crystalline solid at about 25° C. that is malleable. In various embodiments, pliable wax can have a melting point above about 50° C. as measured by USP 741. The pliable wax can have a melting point in a range of about 45° C. to about 100° C., and a penetration value, as measured using ASTM D1321, greater than 40 dmm (decimillimeters) at 25° C. A pliable wax having a penetration value of less than 40 dmm at 25° C. can be too hard to use, so may be avoided.

In various embodiments, the pliable wax can be polyhydroxystearic acid, C₁₈-C₃₈ alkyl hydroxystearoyl stearate, and combinations thereof, which do not form crystalline structures. The C₁₈-C₃₈ alkyl hydroxystearoyl stearate can be a blend of alkyl hydroxystearoyl stearates having chain lengths in the range of C₅-C₃₈.

The pliable wax can be the base for the cosmetic formulation that provides form and texture to the cosmetic, and reduces or prevents the crystallization of a plant derived film former included in the formulation. The pliable wax can be a plasticizer that allows the film former to adhere to a skin surface without flaking or cracking for a period of time. The volatile emollient can vaporize, while the pliable wax(es) and film former(s) remains on the skin surface after application.

In one or more embodiments, the pliable wax can be present in the formulation in a range of about 0.25 weight-percent (wt. %) to about 15.0 wt. %, or about 1.0 wt. % to about 10.0 wt. %, or about 2.0 wt. % to about 8.0 wt. %, although other ranges are also contemplated. In various embodiments, the pliable wax can make up the balance of the cosmetic formulation after all other components are included.

In various embodiments, the plant derived film former can be a hydrophobic polymer, which is derived from a plant, fruit, or vegetable source, which encapsulates actives or colorants in a long wearing and/or water resistant film. The film former can provide long wear, water resistance, and substantivity to the skin.

In various embodiments, the plant derived film former can be glyceryl rosinate, hydrogenated glyceryl abietate, capryloyl glycerin/sebacic acid copolymer, or combinations thereof.

In various embodiments, the plant derived film former can be present in the formulation in a range of about 0.5 wt. % to about 10.0 wt. %, or about 2.0 wt. % to about 7.0 wt. %, or about 3.0 wt. % to about 5.0 wt. %, although other ranges are also contemplated.

In various embodiments, the formulation includes an oil dispersible hyaluronic acid (HA). Hyaluronic Acid is a water soluble, extremely hygroscopic compound that provides hydration and moisturization by binding water and drawing moisture into and on the skin.

In various embodiments, the oil dispersible hyaluronic acid (HA) can be a compound comprised of sodium hyaluronate which is modified using microsphere technology. HA is a non-sulphated glycosaminoglycan (GAG), and is composed of repeating polymeric disaccharides of D-glucuronic acid and N-acetyl-D-glucosamine linked by a glucuronidic β(1→3) bond. The oil dispersible hyaluronic acid (HA) can be uniformly dispersed in an oil matrix and easily delivered to the skin in anhydrous formulations.

In one or more embodiments, the oil dispersible hyaluronic acid can be high molecular weight (HMW) HA having a molecular weight of greater than 1.0 megadaltons (>1.0 M Da), a low molecular weight (LMW) HA having a molecular weight of about 200,000 Daltons (200 K Da) to about 400,000 Daltons (400 K Da), an oligo HA having a molecular weight of less than 10,000 Daltons (Da), or a combination thereof.

In various embodiments, the oil dispersible hyaluronic acid can be present in the formulation in a range of about 0.1 weight-percent (wt. %) to about 2.0 wt. %, or about 0.5 wt. % to about 2.0 wt. %, or about 1.0 weight-percent (wt. %) to about 2.0 wt. %, although other ranges are also contemplated. In various embodiments, no hyaluronic acid may be present in the formulation.

In one or more embodiments, the cosmetic formulation can further include additional emollients, for example, bis-diglyceryl polyacyladipate-2, Persea gratissima (avocado) oil, Ricinus communis (castor) oil, hydrogenated castor oil, and combinations thereof. In various embodiments, Ricinus communis (castor) oil and hydrogenated castor oil can act as a pigment/colorant wetter.

In one or more embodiments, the cosmetic formulation can further include structuring waxes, for example, Euphorbia cerifera (candelilla) wax and/or Oryza sativa (rice) bran wax.

In one or more embodiments, the cosmetic formulation can further include fillers, including, but not limited to, kaolin, mica, and silica. Filler(s) can function as a diluent, but may also provide secondary properties, such as, but not limited to, water or oil absorption, optical blurring, and thickening. In various embodiments, the filler(s) can make up from 0 wt. % to about 80 wt. %, or about 1 wt. % to about 60 wt. %, about 5 wt. % to about 40 wt. %, or about 10 wt. % to about 20 wt. %, or about 20 wt. % to about 40 wt. % of the cosmetic formulation, although other concentrations are also contemplated. In various embodiments, the filler(s) can make up the balance of the cosmetic formulation after all other components are included. F

In one or more embodiments, the cosmetic formulation can further include vitamins, for example, tocopherol (i.e., vitamin E).

In one or more embodiments, the cosmetic formulation can further include water (H₂O) as a diluent.

In one or more embodiments, the cosmetic formulation can further include an emulsifier, for example, disteardimonium hecorite (and) polyglyceryl-2 isostearate (and) polyglyceryl-6 polyricinoleate.

In one or more embodiments, the cosmetic formulation can further include a humectant, for example, glycerin and/or propanediol. A humectant can be hygroscopic ingredients that attract water to the skin to improve hydration levels. Humectants can be included in a formulation in a range of 0 wt. % to about 80 wt. %, about 1 wt. % to about 30 wt. %, or about 1 wt. % to about 10 wt. %, although other ranges are also contemplated.

In one or more embodiments, the cosmetic formulation can further include a stabilizer (e.g., sodium chloride) and/or a preservative (e.g., phenoxyethanol (and) ethylhexylglycerin). A stabilizer is an ingredient that improves immediate and long term stability of the formula, which includes, but is not limited to, emulsifiers, thickeners, electrolytes, and suspending agents. Stabilizer(s) can be included in a formulation in a range of 0 wt. % to about 20 wt. %, about 1 wt. % to about 10 wt. %, or about 1 wt. % to about 5 wt. %, although other ranges are also contemplated.

In one or more embodiments, the cosmetic formulation can further include a thickener, for example, caprylic/capric triglycerides (and) stearalkonium hectorite (and) propylene carbonate. Thickeners include water soluble and oil soluble ingredients that can increase the viscosity of the medium/formulation in which they are dispersed. Thickener(s) can be included in a formulation in a range of 0 wt. % to about 20 wt. %, about 0.01 wt. % to about 20 wt. %, or about 0.05 wt. % to about 10 wt. %, although other ranges are also contemplated.

In one or more embodiments, the cosmetic formulation is a liquid lipstick formulation which applies wet and gradually dries down to a demi matte to matte flexible film. The flexible film can provide even coverage (i.e., uniform film coverage of a skin surface as determined by a clinical grader and/or cosmetologist), all day wear (e.g., 12 hrs., 18 hrs., 24 hrs.), with hydration and moisturization. The flexible film moves with the lips and does not crease, crack, or flake.

In one or more embodiments, the cosmetic formulation of the invention can contain cosmetic colorants, pigments, and/or dyes. The cosmetic colorants, pigments, and/or dyes can include, but are not limited to, iron oxides, titanium dioxide, drug and cosmetic (D&C) and food, drug, and cosmetic (FD&C) dyes, pearls, mica, carmine, fruit oils and extracts, etc.

In one or more embodiments, the cosmetic formulation does not include waxes that are crystalline, for example, carnauba wax, candelilla wax, berry wax, myrica fruit wax, rice bran wax, or sunflower wax.

In one or more embodiments, the cosmetic formulation does not include ceramides.

In one or more embodiments, the cosmetic formulation does not include silicones.

In one or more embodiments, the cosmetic formulation does not include petroleum derived emollients.

In one or more embodiments, the cosmetic formulation does not include a petroleum derived volatile emollient, such as isododecane (i.e., 2,2,4,6,6-Pentamethylheptane), or a silicone derived volatile emollient, for example, cyclopentasiloxane or 1.5 cs dimethicone. or synthetic polymer film former, for example, acrylate or Poly(vinylpolypyrrolidone) (PVP).

In one or more embodiments, the cosmetic formula does not include a synthetic film former derived from silicone, styrene, acrylate or vinylpyrrolidone, such as, but not limited to, trimethylsiloxy silicate, polypropylsilsesquioxane, styrene/acrylates copolymer, acrylates/polytrimethylsiloxymethacrylate copolymer, VP/eicosene copolymer, VP/dimethiconylacrylate/polycarbamyl polyglycol ester, or combinations thereof.

TABLE 1A
EXAMPLE 1, Liquid Lipstick Formulations:

| | | | FORMULA 1 | FORMULA 2 | FORMULA 3 | FORMULA 4 | FORMULA 5 |
|---|---|---|---|---|---|---|---|
| PHASE | INGREDIENT | FUNCTION | (wt. %) | (wt. %) | (wt. %) | (wt. %) | (wt. %) |
| A | BIS-DIGLYCERYL | EMOLLIENT | 10 | 10 | 10 | 10 | 10 |
| | POLYACYLADIPATE-2 | | | | | | |
| A | COCONUT ALKANES (AND) | VOLATILE | 25 | — | 25 | 25 | 25 |
| | COCOCAPRYLATE CAPRATE | EMOLLIENT | | | | | |
| A | UNDECANE (AND) TRIDECANE | VOLATILE | — | 25 | — | — | — |
| | | EMOLLIENT | | | | | |
| B1 | HYDROGENATED METHYL | FILM | 4 | — | — | — | — |
| | ABIETATE | FORMER | | | | | |
| B1 | GLYCERYL ROSINATE | FILM | — | — | 4 | — | — |
| | | FORMER | | | | | |
| B1 | CAPRYLOYL GLYCERIN/ | FILM | — | — | — | 4 | — |
| | SEBACIC ACID COPOLYMER | FORMER | | | | | |
| B1 | PERSEA GRATISSIMA | EMOLLIENT | 4 | 4 | 4 | 4 | 4 |
| | (AVOCADO) OIL | | | | | | |
| B1 | TOCOPHEROL | VITAMIN | 1 | 1 | 1 | 1 | 1 |
| B1 | RICINUS COMMUNIS | PIGMENT | 7 | 7 | 7 | 7 | 7 |
| | (CASTOR) OIL | WETTER | | | | | |
| B1 | CAPRYLIC/CAPRIC | THICKENER | 3 | 3 | 3 | 3 | 3 |
| | TRIGLYCERIDES (AND) | | | | | | |
| | STEARALKONIUM HECTORITE | | | | | | |
| | (AND) PROPYLENE CARBONATE | | | | | | |
| B2 | COLORANTS | COLOR | 15 | 15 | 15 | 15 | 15 |
| C | C18-38 ALKYL | PLASTICIZER | 8 | 8 | 8 | 8 | — |
| | HYDROXYSTEROL STEARATE | | | | | | |
| C | POLYHYDROXYSTEARIC | PLASTICIZER | — | — | — | — | 8 |
| | ACID | | | | | | |
| D | KAOLIN | FUNCTIONAL | 19 | 19 | 19 | 19 | 19 |
| | | FILLER | | | | | |
| D | SILICA | FUNCTIONAL | 3 | 3 | 3 | 3 | 3 |
| | | FILLER | | | | | |
| D | SODIUM HYALURONATE (AND) | MOISTURIZING | 1 | 1 | 1 | 1 | 1 |
| | RICINUS COMMUNIS | ACTIVE | | | | | |
| | (CASTOR) OIL (AND) | | | | | | |
| | HYDROGENATED CASTOR OIL | | | | | | |

TABLE 1B
EXAMPLE 1, Liquid Lipstick Formulation without hyaluronic acid:

| | | | FORMULA 6 |
|---|---|---|---|
| PHASE | INGREDIENT | FUNCTION | (wt. %) |
| A | BIS-DIGLYCERYL | EMOLLIENT | 10 |
| | POLYACYLADIPATE-2 | | |
| A | COCONUT ALKANES | VOLATILE | 25 |
| | (AND) COCO-CAPRYLATE | EMOLLIENT | |
| | CAPRATE | | |
| A | UNDECANE (AND) | VOLATILE | — |
| | TRIDECANE | EMOLLIENT | |
| B1 | HYDROGENATED | FILM FORMER | 4 |
| | METHYL ABIETATE | | |
| B1 | GLYCERYL ROSINATE | FILM FORMER | — |
| B1 | CAPRYLOYL | FILM FORMER | — |
| | GLYCERIN/SEBACIC | | |
| | ACID COPOLYMER | | |
| B1 | PERSEA GRATISSIMA | EMOLLIENT | 4 |
| | (AVOCADO) OIL | | |
| B1 | TOCOPHEROL | VITAMIN | 1 |
| B1 | RICINUS COMMUNIS | PIGMENT | 7 |
| | (CASTOR) OIL | WETTER | |
| B1 | CAPRYLIC/CAPRIC | THICKENER | 3 |
| | TRIGLYCERIDES (AND) | | |
| | STEARALKONIUM | | |
| | HECTORITE (AND) | | |
| | PROPYLENE CARBONATE | | |
| B2 | COLORANTS | COLOR | 15 |
| C | C18-38 ALKYL | PLASTICIZER | — |
| | HYDROXYSTEROL | | |
| | STEARATE | | |
| C | POLYHYDROXYSTEARIC | PLASTICIZER | 8 |
| | ACID | | |
| D | KAOLIN | FUNCTIONAL | 19 |
| | | FILLER | |
| D | SILICA | FUNCTIONAL | 3 |
| | | FILLER | |
| D | SODIUM HYALURONATE | MOISTURIZING | 0 |
| | (AND) RICINUS | ACTIVE | |
| | COMMUNIS (CASTOR) | | |
| | OIL (AND) | | |
| | HYDROGENATED | | |
| | CASTOR OIL | | |

Formulations in Table 1A and Table 1B were prepared by adding Phase A ingredients to a closed kettle with mixing. For Phase B, the B1 ingredients were added to aside kettle and mixed until homogeneous. B2 ingredients were added to B1 and mixed to fully wet out the pigments. Once B1 and B2 were homogeneous, B3 was added and thoroughly mixed in. Phase B was passed three times through a three roll-mill to disperse pigments. Phase B was then added to Phase A in the closed kettle and the kettle was heated to 90° C. with mixing. Phase C was added to the main kettle once the temperature reached 90° C. and mixed until Phase C was fully melted. Phase D ingredients were added and mixed for 10 minutes. To finish the formula, heat and mixing were removed and the batch was left to cool to 25° C. undisturbed.

TABLE 2A
EXAMPLE 2, Lipstick Formulations:

| | | | FORMULA 1 | FORMULA 2 | FORMULA 3 | FORMULA 4 |
|---|---|---|---|---|---|---|
| PHASE | INGREDIENT | FUNCTION | (wt. %) | (wt. %) | (wt. %) | (wt. %) |
| A | RICINUS COMMUNIS | EMOLLIENT | 35 | 35 | 35 | 35 |
| | (CASTOR) OIL | | | | | |
| A | PERSEA GRATISSIMA | EMOLLIENT | 20 | 20 | 20 | 20 |
| | (AVOCADO) OIL | | | | | |
| A | HYDROGENATED METHYL | FILM FORMER | 4 | — | 4 | 4 |
| | ABIETATE | | | | | |
| A | CAPRYLOYL GLYCERIN/ | FILM FORMER | — | 4 | — | — |
| | SEBACIC ACID COPOLYMER | | | | | |
| A | COCONUT ALKANES (AND) | VOLATILE | 2 | 2 | — | 2 |
| | COCOCAPRYLATE CAPRATE | EMOLLIENT | | | | |
| A | UNDECANE AND TRIDECANE | VOLATILE | — | — | 2 | — |
| | | EMOLLIENT | | | | |
| B | EUPHORBIA CERIFERA | STRUCTURING | 4 | 4 | 4 | 5 |
| | (CANDELILLA) WAX | WAX | | | | |
| B | ORYZA SATIVA | STRUCTURING | 9 | 9 | 9 | 9 |
| | (RICE) BRAN WAX | WAX | | | | |
| B | POLYHYDROXY STEARIC | PLASTICIZING | 3 | 3 | 3 | — |
| | ACID | WAX | | | | |
| B | C18-38 ALKYL | PLASTICIZING | — | — | — | 2 |
| | HYDROXYSTEROL STEARATE | WAX | | | | |
| C | RICINUS COMMUNIS | PIGMENT | 9 | 9 | 9 | 9 |
| | (CASTOR) OIL | WETTER | | | | |
| C | COLORANTS | COLOR | 8 | 8 | 8 | 8 |
| D | SILICA | FUNCTIONAL | 6 | 6 | 6 | 6 |
| | | FILLER | | | | |
| D | SODIUM HYALURONATE (AND) | MOISTURIZING | 1 | 1 | 1 | 1 |
| | RICINUS COMMUNIS | ACTIVE | | | | |
| | (CASTOR) OIL (AND) | | | | | |
| | HYDROGENATED CASTOR OIL | | | | | |

TABLE 2B
EXAMPLE 2, Lipstick Formulation without hyaluronic acid:

| | | | FORMULA 5 |
|---|---|---|---|
| PHASE | INGREDIENT | FUNCTION | (wt. %) |
| A | RICINUS COMMUNIS | EMOLLIENT | 36 |
| | (CASTOR) OIL | | |
| A | PERSEA GRATISSIMA | EMOLLIENT | 20 |
| | (AVOCADO) OIL | | |
| A | HYDROGENATED METHYL | FILM FORMER | 4 |
| | ABIETATE | | |
| A | CAPRYLOYL | FILM FORMER | — |
| | GLYCERIN/SEBACIC | | |
| | ACID COPOLYMER | | |
| A | COCONUT ALKANES | VOLATILE | — |
| | (AND) COCO-CAPRYLATE | EMOLLIENT | |
| | CAPRATE | | |
| A | UNDECANE AND | VOLATILE | 2 |
| | TRIDECANE | EMOLLIENT | |
| B | EUPHORBIA CERIFERA | STRUCTURING | 5 |
| | (CANDELILLA) WAX | WAX | |
| B | ORYZA SATIVA (RICE) | STRUCTURING | 9 |
| | BRAN WAX | WAX | |
| B | POLYHYDROXYSTEARIC | PLASTICIZING | — |
| | ACID | WAX | |
| B | C18-38 ALKYL | PLASTICIZING | 2 |
| | HYDROXYSTEROL | WAX | |
| | STEARATE | | |
| C | RICINUS COMMUNIS | PIGMENT | 9 |
| | (CASTOR) OIL | WETTER | |
| C | COLORANTS | COLOR | 8 |
| D | SILICA | FUNCTIONAL | 6 |
| | | FILLER | |
| D | SODIUM HYALURONATE | MOISTURIZING | 0 |
| | (AND) RICINUS COMMUNIS | ACTIVE | |
| | (CASTOR) OIL (AND) | | |
| | HYDROGENATED CASTOR | | |
| | OIL | | |

Formulations in Table 2A and Table 2B were prepared by adding Phase A ingredients to a main kettle with mixing and heating to 90° C. While Phase A was heating, Phase B ingredients were weighed out in aside container. Phase C was prepared by mixing the colorants with the castor oil and processing three times through a three roll mill to disperse the pigments. Phase C was added to the main kettle and mixing and heating was continued. Once Phase A+C reached 90° C., Phase B was added to the main kettle and mixed until all of the waxes were fully melted and dispersed. Phase D ingredients were added to the main kettle and mixed in. Lipstick bullets were then molded while bulk was at 90° C. and chilled to 50° C.

TABLE 3
EXAMPLE 3, Foundation Formulations:

| | | | FORMULA | FORMULA |
|---|---|---|---|---|
| PHASE | INGREDIENT | FUNCTION | 1 (wt. %) | 2 (wt. %) |
| A | DISTEARDIMONIUM | EMULSIFIER | 6 | 6 |
| | HECORITE (AND) | | | |
| | POLYGLYCERYL-2 | | | |
| | ISOSTEARATE (AND) | | | |
| | POLYGLYCERYL-6 | | | |
| | POLYRICINOLEATE | | | |
| A | COCONUT ALKANES | VOLATILE | 22 | 22 |
| | (AND) COCO-CAPRYLATE | EMOLLIENT | | |
| | CAPRATE | | | |
| A | AVOCADO OIL | EMOLLIENT | 5 | 5 |
| B | HYDROGENATED | FILM FORMER | 2 | — |
| | METHYL ABIETATE | | | |
| B | CAPRYLOYL | FILM FORMER | — | 2 |
| | GLYCERIN/SEBACIC | | | |
| | ACID COPOLYMER | | | |
| B | COCO-CAPRYLATE | PIGMENT | 7 | 7 |
| | CAPRATE | WETTER | | |
| B | TITANIUM DIOXIDE | COLOR | 11 | 11 |
| | (AND) LAUROYL LYSINE | | | |
| B | CI 77492 (AND) | COLOR | 3 | 3 |
| | LAUROYL LYSINE | | | |
| B | CI 77491 (AND) | COLOR | 2 | 2 |
| | LAUROYL LYSINE | | | |
| B | CI 77499 (AND) | COLOR | 1 | 1 |
| | LAUROYL LYSINE | | | |
| C | C18-38 ALKYL | PLASTICIZER | 1 | 1 |
| | HYDROXYSTEROL | | | |
| | STEARATE | | | |
| D | MICA | FUNCTIONAL | 6 | 6 |
| | | FILLER | | |
| E | WATER | DILUENT | 25 | 25 |
| E | PHENOXYETHANOL (AND) | PRESERVATIVE | 1 | 1 |
| | ETHYLHEXYLGLYCERIN | | | |
| E | GLYCERIN | HUMECTANT | 3 | 3 |
| E | PROPANEDIOL | HUMECTANT | 4 | 4 |
| E | SODIUM CHLORIDE | STABILIZER | 1 | 1 |

Formulations in Table 3 were prepared by adding Phase A ingredients to the main kettle and mixed with a dispersion blade to fully disperse the hectorite. Phase B ingredients were mixed in a side container and processed three times through a three roll mill to fully disperse the pigments. Phase B was added to Phase A and the batch was mixed and heated to 85° C. with the kettle closed. Once the main kettle reached 85° C., Phase C was added followed by Phase D with mixing and heating. In a separate vessel, Phase E ingredients were combined and heated to 85° C. Phase E was added to the main kettle slowly and the mixing speed was gradually increased as the formula became thicker. Once all of Phase E was added, heat was removed and the batch was mixed until the temperature reached 30° C. The formula was homogenized for 5 minutes to complete the batch.

In various embodiments, the cosmetic formulation provides a hydrating and/or moisturizing formula for all day comfort was supported by a clinical study demonstrating statistically significant improvement of lip hydration and trans-epidermal water loss (TEWL) measurements after 8 hours of wear.

In various embodiments, the cosmetic formulation improves skin barrier function by locking in moisture all day/for up to 8 hours, as supported by a clinical study demonstrating statistically significant improvement of lip TEWL measurements after 8 hours of wear.

In various embodiments, the cosmetic formulation provides all day wear without over drying, as supported by a clinical study demonstrating statistically significant improvement of objectively graded color coverage and lip hydration/TEWL measurements after 8 hours of wear.

Testing of a cosmetic formulation was conducted to determine the duration of wear, lip hydration and trans-epidermal water loss (TEWL). Testing was conducted with subjects that arrived at the clinic with a clean face having removed all makeup in an environmentally controlled room with predetermined temperature and humidity conditions. The subjects were restricted from drinking hot beverages, ingesting caffeine containing products, eating hot, spicy foods, and/or smoking a minimum of 30 minutes (min) before study evaluations. The subjects were evaluated at predetermined intervals.

The formulation set forth in Table 1A, Formula 1, including 10.40 wt. % bis-diglyceryl polyacyladipate-2, 25.00 wt. % coconut alkanes (and) coco-caprylate caprate, 4.00 wt. % hydrogenated methyl abietate, 4.00 wt. % Persea gratissima (avocado) oil, 0.10 wt. % tocopherol, 7.60 wt. % Ricinus communis (castor) oil, 3.00 wt. % caprylic/capric triglycerides (and) stearalkonium hectorite (and) propylene carbonate, 15.00 wt. % colorants, 0.40 wt. % fumed silica (SiO₂), 8.00 wt. % c18-38 alkyl hydroxysterol stearate, 19.00 wt. % kaolin, 2.00 wt. % silica (SiO₂), 1.00 wt. % sodium hyaluronate (and) Ricinus communis (castor) oil (and) hydrogenated castor oil, and 0.50 wt. % hydroxyacetophenone, was the cosmetic formulation used for testing and obtaining the test results herein. Use of the term “(and)” in parenthesis indicates the material can be a mixture of the recited components depending on the source of the component. “Fumed” refers to a synthetic, amorphous silica, also referred to as pyrogenic silica, produced in a flame, which can consist of microscopic droplets of amorphous silica fused into branched, chainlike, three-dimensional secondary particles which then agglomerate into tertiary particles.

Application was performed by a licensed cosmetologist following instructions, where the cosmetologist ensured uniform and consistent application of the test cosmetic formulation for each subject. The test cosmetic formulation was applied to the subjects lips, such that a smooth, even coverage was applied. The test cosmetic formulation was allowed to dry for about 5 minutes after application.

Skin hydration was measured using a Corneometer® CM 825 (Courage and Khazaka electronic GmbH, Germany) by capacitance measurement of the stratum corneum, the uppermost layer of the skin. The measurement can detect changes in the hydration level. Three repeated measurements were taken on the lower lip of the test subjects, not including the moist portion of the lip. The three repeated measurements were taken at each time point of the study.

TABLE 4
Skin Hydration Measurements by Corneometer ®.

| INTERVAL | MEAN | Standard Deviation (SD) |
|---|---|---|
| BASELINE | 29.83 | 7.75 |

| | | | |
|---|---|---|---|
| 15 | Minutes | 42.20 | 6.42 |
| 6 | hours | 38.16 | 6.26 |
| 8 | hours | 35.21 | 6.61 |

Test results were determined by comparing the three repeated measurements to the subject's baseline measurement, where an increase in the measured value indicates an increase in skin hydration. 100% of test subjects still showed improvement in hydration after 6 hours, and 96.67% of test subjects still showed improvement in hydration after 8 hours.

TABLE 5
Skin Hydration Results.

| | 15 Min | 6 Hrs. | 8 Hrs. |
|---|---|---|---|
| Mean % Diff. from Baseline | 41.45% | 27.90% | 18.02% |
| Percent of Subjects with Improvement | 100% | 100% | 96.67% |

Transepidermal Water Loss (TEWL) was measured using a DermaLab® Evaporimeter (Cortex Technology, Hadsund, Denmark). Measurements were also taken on the lower lip of test subjects. Decreases in post barrier disruption indicate an improvement in skin barrier function, such that less water is lost through the skin.

TABLE 6
Mean Skin Barrier Function Values.

| INTERVAL | Mean | Standard Deviation (SD) |
|---|---|---|
| Baseline | 45.81 | 14.01 |
| 6 Hours | 31.68 | 9.86 |
| 8 Hours | 36.64 | 8.72 |

In various embodiments, the cosmetic formulation provides a flexible, all day formula that won't feather, flake or migrate was supported by a clinical study demonstrating statistically significant improvement of objectively graded feathering, flaking, and migration properties and consumer perception of flexibility after 8 hours of wear. The cosmetic was applied to the lips until there was a smooth, even coverage by a licensed cosmetologist and allowed to dry for about 5 minutes. Subjects had a clean face with no make-up. Subjects remained in a temperature controlled room at 20-24 C and 30-50% relative humidity for approximately 8 hours after application. Subjects were age 25 yrs.-50 yrs., and female.

TABLE 7
Long Lasting Color Coverage (Wear)

| | BASE LINE | | | |
|---|---|---|---|---|
| | (Immediately Post- | | | |
| COVERAGE | Application) | 15 Min. | 6 Hrs. | 8 Hrs. |
| Complete Coverage | 100.00% | 100.00% | 46.67% | 33.33% |
| (Score of 3) | | | | |
| Score of 2.5 | 0.00% | 0.00% | 33.33% | 36.67% |
| Moderate Coverage | 0.00% | 0.00% | 16.67% | 13.33% |
| (Score of 2) | | | | |
| Score of 1.5 | 0.00% | 0.00% | 0.00% | 6.67% |
| Mild Coverage | 0.00% | 0.00% | 3.33% | 6.67% |
| (Score of 1.0) | | | | |
| Score of 0.5 | 0.00% | 0.00% | 0.00% | 3.33% |
| No Coverage | 0.00% | 0.00% | 0.00% | 0.00% |
| (Score of 0) | | | | |

TABLE 8
Mean Color Coverage Values.

| INTERVAL | Mean | Standard Deviation (SD) |
|---|---|---|
| Baseline | 3.00 | 0.00 |

| | | | |
|---|---|---|---|
| 15 | Minutes | 3.00 | 0.00 |
| 6 | Hours | 2.60 | 0.48 |
| 8 | Hours | 2.37 | 0.68 |

For the Test, 100.00% of the subjects had complete coverage immediately after application of the cosmetic. 100.00% of the subjects had complete coverage 15 minutes (Min.) after application of the cosmetic. 80.00% of the subjects had a score equal to or greater than 2.5 at 6 hours (Hrs.) after application of the cosmetic.

TABLE 9
Feathering

| | BASE LINE | | | |
|---|---|---|---|---|
| | (Immediately Post- | | | |
| FEATHERING | Application) | 15 Min. | 6 Hrs. | 8 Hrs. |
| No Feathering | 100.00% | 100.00% | 20% | 6.67% |
| (Score of 3) | | | | |
| Score of 2.5 | 0.00% | 0.00% | 63.33% | 43.33% |
| Mild Feathering | 0.00% | 0.00% | 13.33% | 26.67% |
| (Score of 2) | | | | |
| Score of 1.5 | 0.00% | 0.00% | 0.00% | 10.00% |
| Moderate Feathering | 0.00% | 0.00% | 3.33% | 10.00% |
| (Score of 1.0) | | | | |
| Score of 0.5 | 0.00% | 0.00% | 0.00% | 3.33% |
| Severe Feathering | 0.00% | 0.00% | 0.00% | 0.00% |
| (Score of 0) | | | | |

TABLE 10
Mean Feathering Values.

| INTERVAL | Mean | Standard Deviation (SD) |
|---|---|---|
| Baseline | 3.00 | 0.00 |

| | | | |
|---|---|---|---|
| 15 | Minutes | 3.00 | 0.00 |
| 6 | Hours | 2.48 | 0.40 |
| 8 | Hours | 2.08 | 0.62 |

For the Test, 100.00% of the subjects had no feathering immediately after application of the cosmetic. 100.00% of the subjects had no feathering 15 minutes (Min.) after application of the cosmetic. 83.33% of the subjects had a score equal to or greater than 2.5 at 6 hours (Hrs.) after application of the cosmetic.

TABLE 11
Budging/Migration

| | BASE LINE | | | |
|---|---|---|---|---|
| | (Immediately Post- | | | |
| MOVEMENT | Application) | 15 Min. | 6 Hrs. | 8 Hrs. |
| No Movement | 100.00% | 100.00% | 33.33% | 16.67% |
| (Score of 3) | | | | |
| Score of 2.5 | 0.00% | 0.00% | 43.33% | 40.00% |
| Mild Movement | 0.00% | 0.00% | 20.00% | 23.33% |
| (Score of 2) | | | | |
| Score of 1.5 | 0.00% | 0.00% | 0.00% | 0.00% |
| Moderate Movement | 0.00% | 0.00% | 3.33% | 16.67% |
| (Score of 1.0) | | | | |
| Score of 0.5 | 0.00% | 0.00% | 0.00% | 3.33% |
| Severe Movement | 0.00% | 0.00% | 0.00% | 0.00% |
| (Score of 0) | | | | |

TABLE 12
Mean Movement Values.

| INTERVAL | Mean | Standard Deviation (SD) |
|---|---|---|
| Baseline | 3.00 | 0.00 |

| | | | |
|---|---|---|---|
| 15 | Minutes | 3.00 | 0.00 |
| 6 | Hours | 2.52 | 0.46 |
| 8 | Hours | 2.15 | 0.71 |

For the Test, 100.00% of the subjects had no movement immediately after application of the cosmetic. 100.00% of the subjects had no movement 15 minutes (Min.) after application of the cosmetic. 76.67% of the subjects had a score equal to or greater than 2.5 at 6 hours (Hrs.) after application of the cosmetic.

TABLE 13
Flaking

| | BASE LINE | | | |
|---|---|---|---|---|
| | (Immediately Post- | | | |
| FLAKING | Application) | 15 Min. | 6 Hrs. | 8 Hrs. |
| No Flaking | 100.00% | 96.67% | 73.33% | 46.67% |
| (Score of 3) | | | | |
| Score of 2.5 | 0.00% | 3.33% | 20.00% | 40.00% |
| Mild Flaking | 0.00% | 0.00% | 3.33% | 6.67% |
| (Score of 2) | | | | |
| Score of 1.5 | 0.00% | 0.00% | 0.00% | 0.00% |
| Moderate Flaking | 0.00% | 0.00% | 3.33% | 6.67% |
| (Score of 1.0) | | | | |
| Score of 0.5 | 0.00% | 0.00% | 0.00% | 0.00% |
| Severe Flaking | 0.00% | 0.00% | 0.00% | 0.00% |
| (Score of 0) | | | | |

TABLE 14
Mean Flaking Values.

| INTERVAL | Mean | Standard Deviation (SD) |
|---|---|---|
| Baseline | 3.00 | 0.00 |

| | | | |
|---|---|---|---|
| 15 | Minutes | 2.98 | 0.09 |
| 6 | Hours | 2.80 | 0.43 |
| 8 | Hours | 2.60 | 0.53 |

For the Test, 100.00% of the subjects had no flaking immediately after application of the cosmetic. 96.67% of the subjects had no flaking 15 minutes (Min.) after application of the cosmetic. 100.00 of the subjects had a score greater than 2.5 at 15 minutes (Min.) after application of the cosmetic. 93.33% of the subjects had a score equal to or greater than 2.5 at 6 hours (Hrs.) after application of the cosmetic.

86.67% of the subjects felt that the cosmetic felt flexible on their lips at 15 Min. and 80.00% felt that the cosmetic felt flexible on their lips at 6 hrs.

Having described preferred embodiments of a cosmetic formulation and method of preparing the cosmetic formulations (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

What is claimed is:

1. A cosmetic formulation, comprising:
a plant derived volatile emollient;
a plant derived film former; and
a pliable wax, wherein the volatile emollient, film former and pliable wax are combined to provide an increased skin hydration for at least 6 hours compared to a baseline skin hydration without application of a cosmetic.

2. The cosmetic formulation of claim 1, wherein the plant derived volatile emollient is an emollient derived from a plant, fruit, or vegetable source with a vapor pressure greater than 0.13 Pascal (Pa) and a boiling point below 230° C.

3. The cosmetic formulation of claim 2, wherein the plant derived volatile emollient is one selected from the group of compounds consisting of coconut alkanes, undecane (C₁₁H₂₄), dodecane (C₁₂H₂₆), tridecane (C₁₃H₂₈), and combinations thereof.

4. The cosmetic formulation of claim 1, wherein the plant derived film former is a hydrophobic polymer derived from a plant, fruit, or vegetable source.

5. The cosmetic formulation of claim 4, wherein the plant derived film former is one selected from a group consisting of glyceryl rosinate, hydrogenated glyceryl abietate, capryloyl glycerin/sebacic acid copolymer, and combinations thereof.

6. The cosmetic formulation of claim 1, wherein the pliable wax is an organic compound or mixture of organic compounds that is a non-crystalline solid at about 25° C., and has a melting point in a range of about 45° C. to about 100° C.

7. The cosmetic formulation of claim 6, wherein the pliable wax is one selected from a group consisting of polyhydroxystearic acid, C₁₈-C₃₈ alkyl hydroxystearoyl stearate, and combinations thereof, which do not form crystalline structures.

8. The cosmetic formulation of claim 1, wherein the plant derived film former is present in the cosmetic formulation in a range of about 0.5 weight percent (wt. %) to about 10.0 wt. %.

9. The cosmetic formulation of claim 8, wherein the plant derived volatile emollient is present in the cosmetic formulation in a range of about 1.0 weight-percent (wt. %) to about 80.0 wt. %.

10. The cosmetic formulation of claim 9, wherein the pliable wax is present in the cosmetic formulation in a range of about 0.25 weight-percent (wt. %) to about 15.0 wt. %

11. The cosmetic formulation of claim 1, further comprising an oil dispersible hyaluronic acid (HA), wherein the hyaluronic acid is a high molecular weight (HMW) HA having a molecular weight of greater than 1.0 megadaltons (>1.0 M Da).

12. A cosmetic formulation, comprising:
a plant derived volatile emollient, wherein the plant derived volatile emollient is an emollient derived from a plant, fruit, or vegetable source with a vapor pressure greater than 0.13 Pa;
a plant derived film former;
a pliable wax, wherein the pliable wax is an organic compound or mixture of organic compounds that is a non-crystalline solid at about 25° C.; and
an oil dispersible hyaluronic acid,
wherein the volatile emollient, film former, pliable wax and hyaluronic acid are combined to form the cosmetic formulation that provides increase skin hydration for at least 6 hours compared to a baseline skin hydration without application of a skin cosmetic.

13. The cosmetic formulation of claim 12, further comprising a colorant, wherein the colorant is one selected from a group consisting of a drug and cosmetic (D&C) or food, drug, and cosmetic (FD&C) dye.

14. The cosmetic formulation of claim 12, wherein the plant derived volatile emollient has a flash point below 100° C.

15. The cosmetic formulation of claim 12, wherein the plant derived volatile emollient has a a boiling point below 230° C.

16. The cosmetic formulation of claim 12, wherein the plant derived volatile emollient is present in the formulation in a range of 10.0 wt. % to about 70.0 wt. %.

17. The cosmetic formulation of claim 12, wherein the pliable wax has a melting point in a range of about 45° C. to about 100° C.

18. The cosmetic formulation of claim 12, wherein the pliable was has a penetration value, as measured using ASTM D1321, greater than 40 dmm (decimillimeters) at 25° C.

19. A cosmetic formulation, comprising:
a plant derived volatile emollient, wherein the plant derived volatile emollient is one selected from a group of coconut alkanes, undecane (C₁₁H₂₄), dodecane (C₁₂H₂₆), tridecane (C₁₃H₂₈), and combinations thereof;
a plant derived film former, wherein the plant derived film former is one selected from a group of glyceryl rosinate, hydrogenated glyceryl abietate, capryloyl glycerin/sebacic acid copolymer, and combinations thereof;
a pliable wax, wherein the pliable wax is one selected from a group consisting of polyhydroxystearic acid, C₁₈-C₃₈ alkyl hydroxystearoyl stearate, and combinations thereof; and
an oil dispersible hyaluronic acid.

20. The cosmetic formulation of claim 19, wherein the plant derived film former is present in the cosmetic formulation in a range of about 2.0 wt. % to about 7.0 wt. %.

21. The cosmetic formulation of claim 19, wherein the pliable wax is present in the cosmetic formulation in a range of about 1.0 wt. % to about 10.0 wt. %.

22. The cosmetic formulation of claim 19, wherein the plant derived volatile emollient is present in the cosmetic formulation in a range of about 1.0 wt. % to about 80.0 wt. %.

23. The cosmetic formulation of claim 19, wherein the cosmetic formulation further comprises castor oil and a colorant, wherein the cosmetic formulation provides increased lip hydration for at least 6 hours compared to a baseline lip hydration without application of a lip cosmetic.
